(19) United States
(12) Patent Application Publication
(10) Pub. No.: US 2017/0231548 A1
(43) Pub. Date: Aug. 17, 2017

(54) SYSTEM AND METHOD FOR BLADDER TRANSDUCER PLACEMENT

(71) Applicant: Indiana University Research and Technology Corporation, Indianapolis, IN (US)

(72) Inventor: Charles R. Powell, Zionsville, IN (US)

(73) Assignee: Indiana University Research and Technology Corporation, Indianapolis, IN (US)

(21) Appl. No.: 15/518,732

(22) PCT Filed: Oct. 29, 2015
(86) PCT No.: PCT/US2015/058100

§ 371 Date: Apr. 12, 2017

Related U.S. Application Data
(60) Provisional application No. 62/069,966, filed on Oct. 29, 2014.

(51) Int. Cl. A61B 5/20; A61B 5/07; A61B 5/00

(52) U.S. Cl. CPC A61B 5/205; A61B 5/6874; A61B 5/076; A61B 5/6882; A61B 2560/0219; A61B 2560/063; A61B 2562/0247

(57) ABSTRACT

A tissue sensing device and a method for placing the device. The device including a first electrically conductive needle; a non-conductive sheath receiving the first needle therein and allowing exposure of a portion the first needle at a distal end thereof; a second electrically conductive needle; and electronics coupled to the first and second needle, the electronics providing for sensing of capacitance between the first and second needles so as to provide an indication of a tissue in which the exposed portion of the first needle is located.

### CROSS-REFERENCE TO RELATED APPLICATIONS

This application claims priority to U.S. Provisional Application Ser. No. 62/069,966, filed on Oct. 29, 2014, the entire disclosure of which is hereby expressly incorporated by reference.

### GOVERNMENT RIGHTS

This invention was made with government support under RR025761 awarded by National Institutes of Health. The government has certain rights in the invention.

### FIELD

The present disclosure relates generally to systems and methods of placing transducers within the body. The present disclosure relates more specifically to systems and methods for placing pressure transducers in an animal or human bladder.

### BACKGROUND

Patients suffering from spinal cord and brain injuries often have significant bladder problems. These include urinary incontinence, urinary retention, urinary tract infections, stone formation in the bladder, and pressure sores from constant wet skin and immobility. Moreover, urinary incontinence is a significant factor leading to the need for skilled caregivers after a spinal cord injury. In some cases, neurogenic bladder problems can lead to kidney failure, urosepsis, and death. As early as 1993 it was recognized that the death rate from sepsis after a spinal cord injury was 82 times higher than expected based on age-matched controls. High pressures in the bladder contribute to damage to the urinary system, renal failure, and often cause incontinence. Standard of care involves measuring these pressures periodically in spinal cord injured patients to assess the risk for kidney deterioration and to find ways to improve incontinence and quality of life.

Prior devices have been embedded under the bladder wall, but this has not been demonstrated in humans. Prior work has demonstrated that embedded devices have a propensity to extrude out into the bladder with time. This has been demonstrated in dogs and goats previously. For purposes of temporary diagnostic testing, tunneling the sensor under the bladder wall would be more invasive than necessary.

Unfortunately no long term conventional method exists to constantly measure these pressures. It is necessary to make periodic bladder pressure measurements in the physician's office, as this equipment is not portable. This practice is known as urodynamic testing. For many of these patients transportation is a hardship. An implantable, wireless device to measure bladder pressure in the comfort of the patient's home would eliminate some of this hardship and improve detection of harmful pressure levels in the bladder.

There are other important reasons to measure bladder pressure. Stimulating the nerves of the bladder, or “neuromodulation,” has been used to treat urinary incontinence in the US and Europe since 1993, but has never been able to incorporate real time bladder pressure data to modify the stimulation because of limitations in technology. This may be one reason that up to 54% of patients successfully treated with neuromodulation still have some incontinence episodes. Accordingly, the ability to measure real time pressure in the bladder is needed, which may make long-term, individualized, patient-responsive neuromodulation of the bladder possible.

### SUMMARY

One aspect of some embodiments is a pressure sensor that can be inserted in the bladder or attached to the wall of the bladder with one or more means for fixating the device. In some embodiments, the device can be released by actuating a means for releasing the device. In some embodiments the releasing means includes one or more flexible, biocompatible lines attached to a pincher or clamp that causes the pincher or clamp to let go of the bladder when actuated.

Still further embodiments include deploying sensors using minimally invasive techniques, and can be injected through a needle from the surface of the abdomen into just beneath the mucosal lining of the bladder where it will sense pressures without being exposed to the urine. A needle sensor may be used in some embodiments to detect the precise location to inject the sensor.

It will be appreciated that the various apparatus and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

The above mentioned and other features and objects of this disclosure, and the manner of attaining them, will become more apparent and the disclosure itself will be better understood by reference to the following description of an embodiment of the disclosure taken in conjunction with the accompanying drawings, wherein:

FIG. 1 is a perspective drawing of a MEMS wireless bladder pressure sensor according to one embodiment of the present invention;

FIGS. 2A-B are views of apparatus for injecting a bladder pressure sensor lead according to an embodiment of the present invention;

FIGS. 3A-D are a schematic, pictorial representation of a method according to another embodiment of the present invention; and

FIG. 4 is a flowchart describing the method illustrated in FIGS. 3A-D.

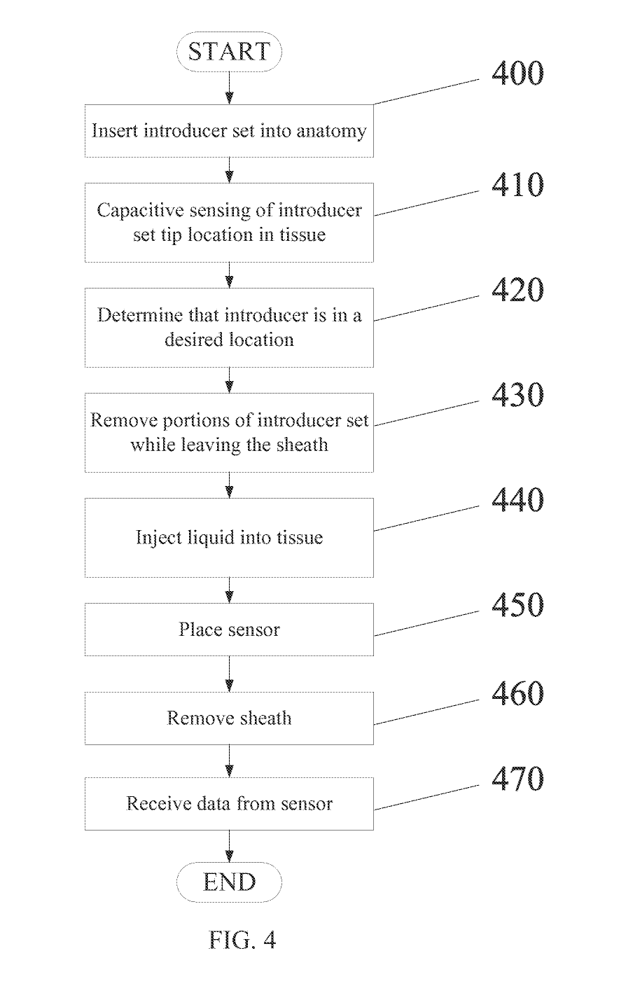

Corresponding reference characters indicate corresponding parts throughout the several views. Although the drawings represent embodiments of the present disclosure, the drawings are not necessarily to scale and certain features may be exaggerated in order to better illustrate and explain the present disclosure. The exemplification set out herein illustrates a embodiments of the disclosure, in one form, and such exemplifications are not to be construed as limiting the scope of the disclosure in any manner.

### DETAILED DESCRIPTION

For the purposes of promoting an understanding of the principles of the invention, reference will now be made to the various embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention. It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims. The usage of words indicating preference, such as “preferably,” refers to features and aspects that are present in at least one embodiment, but which may be optional for some embodiments.

As used herein, the modifier “about” used in connection with a quantity is inclusive of the stated value and has the meaning dictated by the context (for example, it includes at least the degree of error associated with the measurement of the particular quantity). When used in the context of a range, the modifier “about” should also be considered as disclosing the range defined by the absolute values of the two endpoints. For example, the range “from about 2 to about 4” also discloses the range “from 2 to 4.”

Furthermore, with discussion pertaining to a specific composition of matter, that description is by example only, and does not limit the applicability of other species of that composition, nor does it limit the applicability of other compositions unrelated to the cited composition.

A device in some embodiments is fixed to the bladder wall to prevent the device being expelled with the first void. One aspect of this is the method and apparatus by which the device is placed and stays in the bladder. There are several mechanisms by which to fix the device to the bladder wall, including some that would keep it in the bladder but not allow it to be expelled while the subject voids.

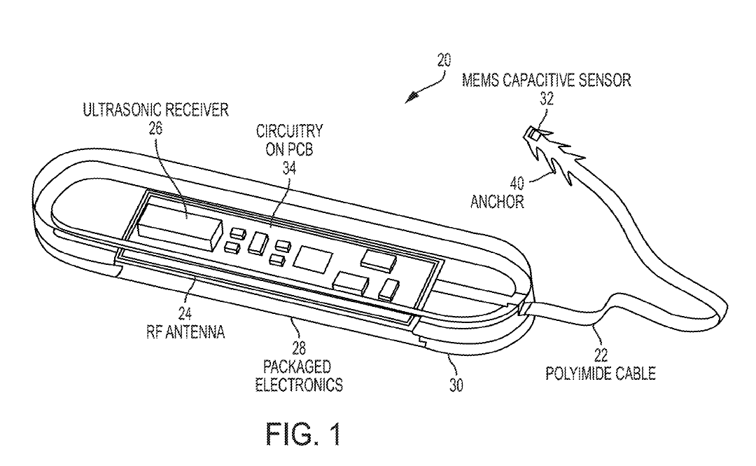

As shown in FIG. 1, a pressure sensor 32 is provided that is suitable for placement within the bladder.

Sensing device 20 for chronic bladder pressure measurement incorporates several aspects that can impact management of patients with urinary incontinence. FIG. 1 shows an exemplary embodiment. As illustrated, sensing device 20 includes pressure sensor 32, flexible interconnect (tethering arm) 22, exemplified as polyimide cable, anchor 40, circuit board 34, and housing 30.

### Pressure Sensor and Flexible Interconnect

One embodiment uses a MEMS capacitive pressure sensor 32. As one example, the device can use a Protron Microteknic (commercially available from Protron Mikrotechnik HmbG) capacitive pressure sensor which has a small footprint (1.2 mm×0.6 mm×0.5 mm) and other characteristics (dynamic range; 0-250 mmHg, and accuracy; ±0.76 mmHg) suitable for the present application (bladder pressure dynamic range; 0-180 mmHg, accuracy; 1 mmHg). The pressure sensor 32 can be flip-chip bonded to a polyimide interconnect cable 22 and coated with parylene (e.g., 5-10 μm) before implantation. In various embodiments, the total thickness of the sensor and substrate may be less than about 1 mm (e.g., about 0.5 mm sensor thickness and between about 0.3 mm and about 0.5 mm polyimide thickness). Sensing device 20, specifically interconnect 22, may include a series of backward-oriented tines/barbs 40 at the tip of the polyimide cable in order to anchor the device to the bladder wall. Barbs 40 are illustratively created using laser micromaching (e.g., a CO₂ laser). The number and shape of the barbs needed to securely anchor the sensor on the bladder wall can be other than that exemplified in the figures. Interconnect 22 illustratively is electronically coupled to circuit board 34 such that pressures sensed by sensor 32 and converted into electrical signals are transmitted to electronics capable of making use of such data.

Sensor 32 and interconnect 22 may be coupled to circuit board 34 (that can be placed in or remain outside of the body but provide wireless monitoring) or to electronics (e.g., packaged electronics 28) configured to be located outside of the body and maintain a wired connection thereto.

### Circuit Board and Housing

In various wireless embodiments, sensing device 20 illustratively may use ultrasound to power itself, including the transponder (about 20 mm×5 mm×5 mm). This allows penetration deep into the tissue (about 20 to about 30 cm). Ultrasound penetration depth depends on the frequency and for our application, and poses a trade-off between the depth and the ultrasonic receiver dimensions. Various embodiments may use 2.15 MHz and can power the device at depths of about 10-20 cm with almost omni-directional performance. The ultrasound omni-directionality compares to inductive methods that require a good alignment between the transmitter and receiver coils. The alignment insensitivity in ultrasound is due to reflections of the radiated power at the tissue air boundaries which feeds back the signal onto the receiver. One proposed implant location allows an ultrasonic transmitter to be positioned over the bladder without any bony prominence or air cavity in between. In various embodiments, the ultrasonic receiver 26 can use either lead zirconate titanate (PZT) or quartz. Sensing device 20 uses a poly(methyl methacrylate) (PMMA), for example PLEXIGLAS, package housing 30 for the proposed transponder in order to protect the electronics. The space inside the package (between the ultrasonic receiver and PMMA wall) includes a polymeric matching layer to reduce the reflections due to acoustic mismatch. Furthermore, sensing device 20 includes an RF antenna that provides for the wireless output of a signal indicative of the pressures sensed by sensor 32.

A received rectified ultrasonic signal is used to generate a DC voltage which is then used to supply a low frequency (e.g., 5-10 kHz) oscillator. The oscillator output feeds an n-type metal-oxide-semiconductor and p-type metal-oxide-semiconductor switch pair which sequentially connect and disconnect the sensing capacitor to the supply. An on-board inductor acting as the transmitter antenna (such as RF antenna 24 in FIG. 1) is connected in parallel with the sensing capacitor through one of the switches. In one cycle when the oscillator output is high, the supply is used to charge the capacitor and in the next cycle the capacitor is disconnected from the supply and connected to the inductor, thus dumping the charge into a parallel LC circuit. The system therefore transmits an RF pulse, frequency of which is a function of the sensor capacitance. The signal can be picked up outside body with an antenna.

The above-described sensing device 20 is exemplary of the type of device that can be placed in the bladder. Furthermore, sensing device 20 is exemplary of a sensing device that can be placed in the bladder by the method described below.

### Introducer and Placement

As previously noted, sensing device 20 and similar devices can be implanted above the bladder. This can be deep to the fascia of the anterior abdominal wall in the space of Retzius so that it is not palpable to the patient, but not in danger of migrating into the bladder.

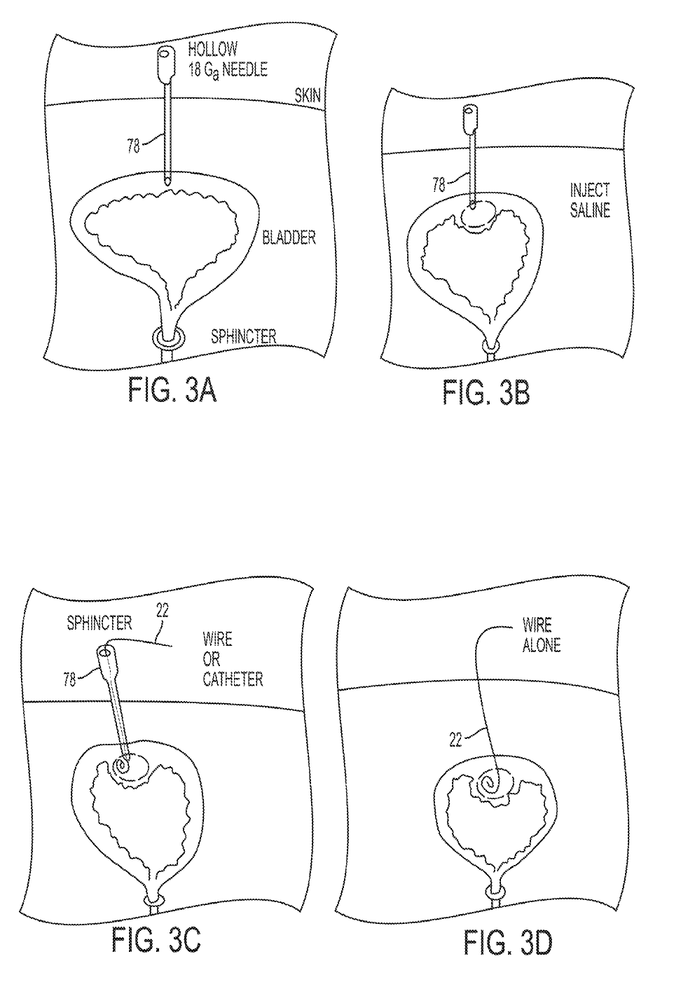

In some embodiments the bulk of the sensor (everything within housing 30) may be placed outside the bladder. Sensing device 20 can be fixed to the fascia. A flexible wire lead is attached to it (such as interconnect cable 22), encapsulated in medical grade silicone, as seen in FIG. 1. This lead 22 terminates in pressure sensor 32 that is tunneled beneath the pubic symphysis into a pocket of bladder mucosa created by injecting saline between the bladder mucosa and detrusor muscle, as seen in FIG. 3.

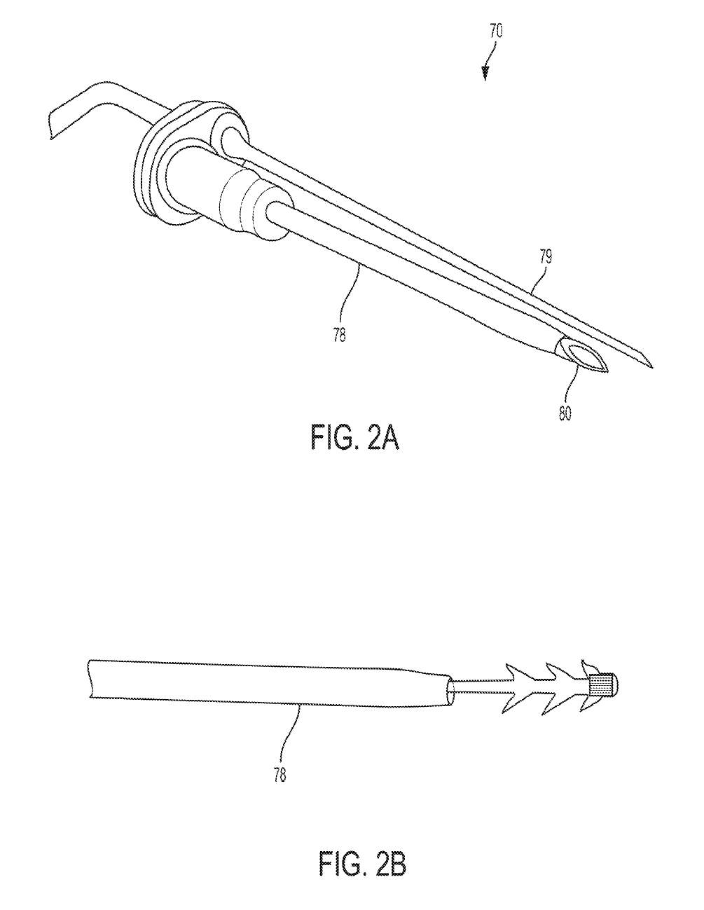

FIG. 2 shows an introducer set 70 operable to facilitate the placement of sensing device 20 in the anatomy. The introducer set 70 includes sheath 78, probe 79, and introducer needle 80. In use, needle introducer sheath 78 is placed through the abdominal wall into the wall of the bladder. Introducer sheath 78 is stopped at the precise point where the bladder muscle joins with the inner mucosal lining of the bladder.

Sheath 78 receives introducer needle 80 therein. The combination of needle 80 and probe 79 provide a capacitive sensing apparatus that enable a user to locate the interface of the bladder muscle and the inner mucosal lining. Sheath 78 illustratively is formed from a material having poor electrical conduction. Sheath 78 is illustratively constructed from polytetrafluoroethylene (PTFE), such as TEFLON, or another similar biocompatible non-conducting material. Needle 80 and probe 79, however, are illustratively formed from steel and has good electrical conductive properties. Introducer set 70 is illustratively a bipolar needle electrode. Probe 79 is coupled to an LCR meter (inductance, capacitance, resistance meter). In various embodiments, needle 80 and probe 79 are physically linked at proximal ends thereof while remaining electrically isolated from each other. Accordingly, probe 79 and needle 80 move as one such that advancing one in anatomy similarly advances the other.

Introducer needle 80 is placed within sheath 78 such that only a portion thereof is exposed out a distal end of sheath 78. The combination of probe 79 and sheath 78 is advanced into and through tissue, FIG. 3A, block 400. A 1000 Hz signal (other frequencies could be used as appropriate for various tissues) is coupled to introducer set 70 to measure capacitance using the bipolar needle electrode, block 410. The conductance of needle 80 and relative non-conductance of sheath 78 combine to provide that capacitance detected by introducer set 70 is indicative of capacitance at a distal end of needle 80 rather than all along its length within tissue. Accordingly, sensed capacitance is indicative of the tissue encountered by the distal end of needle 80.

From in-vitro testing, capacitance of relevant tissues can be found in Table 1 below. Where each cell in the body acts as a small capacitor, significant discrimination can be achieved between different tissues. A mathematical underpinning for this location technique using the following equation:

$$C = {ɛ}_{r} {ɛ}_{0} \frac{A}{d}$$

where C is the capacitance; this will vary in a predictable way depending on the tissue needle is in, A is the area of overlap of two capacitive plates of the sensor (which is constant based on configuration of needle electrode), εᵣ is the relative static permittivity (sometimes called the dielectric constant) of the material between the plates (accordingly, this value varies based upon the material in which the distal tip of probe 79 is located, for a vacuum, εᵣ=1); ε₀ is the electric constant (εₒ≈8.854×10⁻¹² F m⁻¹); and d is the separation between the plates (which is constant based on configuration of needle electrode). Accordingly, the overall capacitance output by introducer set 70 varies directly according to the tissue the needle 80 and probe 79 are passing through (as a function of εᵣ). Thus, by reading the capacitance indicated by introducer set 70, the type of tissue in which the tip of needle 80 is located can be determined and, more specifically, the interface between bladder muscle and inner mucosal lining can be determined.

TABLE 1
Capacitance of different tissues as sensed by the needle electrode.

| | CAPACITANCE | | |
|---|---|---|---|
| | air | muscle | saline |
| mean | 0.022588235 | 72.04162 | 90.851 |
| min | 0.022 | 51.393 | 74.7 |
| max | 0.027 | 93.99 | 107.5 |
| stdev | 0.00091129 | 11.30214 | 8.446965 |

Once the desired tissue space is located, block 420, needle 80 and probe 79 are retracted from the tissue while leaving sheath 78 in place, block 430. It should be appreciated that, so placed, sheath 78 provides a pathway from extra-corporeal space directly to the junction of bladder muscle and the inner mucosal lining.

Next, the junction of bladder muscle and the inner mucosal lining is infiltrated with sterile water or saline through a lumen of sheath 78 to create a potential space allowing sensor 32 to be advanced without being damaged, FIG. 3B and block 440. The sensor is pushed, or injected, down the sheath 78 to a pre-determined point so that sensor 32 and a portion of interconnect 22 is exposed from the tip of sheath 78 and within the potential space created by the saline, FIG. 3C and block 450. Sheath 78 is then withdrawn, leaving sensor 32 and the interconnect 22 behind in the bladder wall, FIG. 3D and block 460. Tines 40 are deployed as the sheath is withdrawn, fixing sensor 32 and interconnect 22 in the muscle of the bladder, but beneath the inner mucosal surface of the bladder, protecting it from exposure to urine inside the bladder.

As previously noted, placement of sensor 32 is then usable as part of a wireless sensor or with wired components located outside of the body. Thus, the present disclosure generally discloses a device and method of locating areas of body tissues and of placing devices (such as sensor 32) at the located areas.

In some embodiments, such as those where the electronics are located outside the body, interconnect 22 is not yet coupled to circuit board 34 and the components thereon (or other electronics). Accordingly, once sheath 78 is removed from tissue and from interconnect 22, interconnect 22 is coupled to the desired electronics capable of receiving data provided by sensor 32.

With the sensor so placed, data is then received therefrom to provide information about the tissue in which sensor 32 is located, block 470.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.”

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. §112(f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. A tissue sensing device including:
a first electrically conductive needle;
a non-conductive sheath receiving the first needle therein and allowing exposure of a portion the first needle at a distal end thereof;
a second electrically conductive needle; and
electronics coupled to the first electrically conductive needle and second electrically conductive needle, the electronics providing for sensing of capacitance between the first electrically conductive needle and second electrically conductive needle so as to provide an indication of a tissue in which the exposed portion of the first needle is located.

2. The tissue sensing device of claim 1, further comprising a transmitter.

3. The tissue sensing device of claim 2, wherein the transmitter is an RF antenna.

4. The tissue sensing device of claim 2, wherein the transmitter comprises part of the electronics coupled to the first electrically conductive needle and the second electrically conductive needle.

5. The tissue sensing device of claim 1, wherein the non-conductive sheath comprises polyimide.

6. The tissue sensing device of claim 1, wherein the electronics comprises an ultrasonic receiver.

7. The tissue sensing device of claim 6, wherein the ultrasonic receiver is configured to receive an ultrasonic signal and generate a direct current (DC) voltage.

8. A tissue sensor kit comprising:
the tissue sensing device of claim 1;
an introducer needle;
a probe; and
and a introducer sheath configured to receive the tissue sensing device.

9. The tissue sensor kit of claim 8, wherein the probe is coupled to an inductance, capacitance, resistance (LCR) meter.

10. The tissue sensor kit of claim 8, wherein the introducer needle and the probe are coupled at proximal ends thereof.

11. The tissue sensor kit of claim 10, wherein the introducer needle is electrically isolated from the probe.

12. A method of placing a sensor in tissue including:
placing a sheath into a tissue;
sensing a capacitance of at least one needle associated with the sheath;
determine that the sensed capacitance indicated that a distal end of the sheath is in a desired location;
injecting liquid through the sheath; and
sending a sensor through the sheath to the desired location.

13. The method of claim 12, wherein the tissue is a bladder.

14. The method of claim 12, further comprising receiving data from the sensor.

15. The method of claim 12, wherein the sensor comprises;
a first electrically conductive needle;
a non-conductive sheath receiving the first needle therein and allowing exposure of a portion the first needle at a distal end thereof;
a second electrically conductive needle; and
electronics coupled to the first and second needle, the electronics providing for sensing of capacitance between the first and second needles so as to provide an indication of a tissue in which the exposed portion of the first needle is located.

16. The method of claim 12, further comprising removing a portion of the sheath.

17. The method of claim 13, wherein sheath provides a pathway from extra-corporeal space directly to a junction of a muscle of the bladder and an inner mucosal lining

18. The method of claim 15, wherein the electronics are located outside of a body.

19. The method of claim 18, further comprising removing the sheath.

20. The method of claim 19, further comprising electrically coupling the electronics to the first electrically conductive needle after removing the sheath.
